(19) United States
(12) Patent Application Publication
(10) Pub. No.: US 2020/0255838 A1
(43) Pub. Date: Aug. 13, 2020

(54) Selenium Cancer Therapy

(71) Applicant: University of Massachusetts, Boston, MA (US)

(72) Inventors: Dohoon Kim, Shrewsbury, MA (US); Anne E. Carlisle, Worcester, MA (US); Namgyu Lee, Worchester, MA (US)

(21) Appl. No.: 16/642,479

(22) PCT Filed: Sep. 7, 2018
(86) PCT No.: PCT/US18/50082

§ 371 Date: Feb. 27, 2020

Related U.S. Application Data
(60) Provisional application No. 62/555,310, filed on Sep. 7, 2017.

(51) Int. Cl. C12N 15/113; C12N 9/22; G01N 33/68; G01N 33/497; A61P 35/00

(52) U.S. Cl. CPC C12N 15/1137; C12N 9/22; G01N 33/6848; C12N 2310/14; A61P 35/00; C12Y 207/09003; C12N 2310/20; G01N 33/497

(57) ABSTRACT

Methods in which targeted SEPHS2 disruption/inhibition and/or administration of selenite are used, e.g., in cancers that express SLC7A11.

### CLAIM OF PRIORITY

This application claims the benefit of U.S. Application No. 62/555,310, filed on Sep. 7, 2017. The entire contents of the foregoing are hereby incorporated by reference.

### TECHNICAL FIELD

Described herein are methods for treating cancer in which targeted selenophosphate synthetase 2 (SEPHS2) disruption/inhibition and/or administration of selenite are used, e.g., in cancers that express solute carrier family 7 member 11 (SLC7A11).

### BACKGROUND

Selenium is a trace nutrient that must be obtained in small quantities from the diet. As its main role, inorganic forms of selenium are incorporated via the selenocysteine biosynthesis pathway to produce selenocysteinyl-tRNA which is itself incorporated in the translation of selenoproteins, a group of 25 proteins which include redox enzymes such as glutathione peroxidases, and thioredoxin reductases. Thus, the production of a subset of proteins in our body requires selenium.

On the other hand, ingestion of high doses of selenium in various inorganic forms such as selenite and selenate can be toxic and thus selenium has been long recognized as an environmental toxin, found in high quantities in certain plants in selenium-rich regions. Subsequent studies have reported¹ (in 1966) that radioactive selenite, a dietary form of selenium, accumulates in tumors, and studies in cancer cell culture and xenograft models have shown that selenite and other forms of selenium can exert toxicity to cancer cells and can impair tumor growth. Others have described the use of inorganic selenium (selnate and selenite, WO 2006032074) and selenium nanoparticles (US 20110262564). Furthermore, there are completed and ongoing clinical trials that involve sodium selenite or other forms of selenium to treat cancer, either alone or in conjunction with existing therapeutic strategies.

### SUMMARY

Some evidence indicates that cancer cells have increased uptake of selenium compared to normal cells¹. Described herein are findings that identify a mediator of selenium entry into cells, SLC7A11, which is also increased in expression in several cancer types. Furthermore, we link SLC7A11 and increased selenium entry in cancer cells to an increase in selenoprotein expression for important redox proteins such as glutathione peroxidase 1, suggesting for the first time both a mechanism and a functional purpose for upregulated selenium metabolism in cancer.

Furthermore, we show for the first time a critical detoxifying role of selenocysteine biosynthesis pathway, in particular the enzyme SEPHS2, in processing toxic inorganic selenium compounds into nontoxic selenoproteins. Thus, the detoxification of toxic selenium compounds by SEPHS2 is a critical determinant of the toxic/effective window for selenium.

Thus, provided herein are methods for treating a cancer in a subject. The methods include administering to the subject an inhibitor of SEPHS2, wherein the inhibitor of SEPHS2 is an inhibitory nucleic acid, e.g., an antisense, siRNA, or LNA targeting a SEPHS2 nucleic acid, or a CRISPR/Cas9 complex targeting a SEPHS2 gene.

In some embodiments, the CRISPR/Cas9 complex targeting a SEPHS2 gene is delivered via AAV or as a ribonucleoprotein complex.

In some embodiments, the cancer is a brain cancer, breast cancer, or renal cancer.

In some embodiments, the methods include administering a treatment comprising administration of selenite or to the subject.

Also provided herein are methods for determining whether a subject who has cancer is likely to respond to a treatment comprising administration of selenite, and optionally selecting a subject who has cancer for treatment with selenite. The methods include determining a level of SLC7A11 expression in a sample comprising cancer cells from the subject; comparing the level of SLC7A11 in the sample to a reference level, wherein the presence of a level of SLC7A11 in the sample above the reference level indicates that the subject is likely to respond to a treatment comprising administration of selenite; and optionally selecting the subject for a treatment comprising administration of selenite. In some embodiments, the methods include administering a treatment comprising administration of selenite to the subject who has a level of SLC7A11 in the sample above the reference level.

In some embodiments, the methods include administering to the subject an inhibitor of SEPHS2, wherein the inhibitor of SEPHS2 is an inhibitory nucleic acid, e.g., an antisense, siRNA, or LNA targeting a SEPHS2 nucleic acid, or a CRISPR/Cas9 complex targeting a SEPHS2 gene.

In some embodiments, the cancer is a brain cancer, renal cancer, or breast cancer.

Also provide herein are inhibitors of SEPHS2, and compositions comprising the same, wherein the inhibitor of SEPHS2 is an inhibitory nucleic acid, e.g., an antisense, siRNA, or LNA targeting a SEPHS2 nucleic acid, or a CRISPR/Cas9 complex targeting a SEPHS2 gene, for use in treating cancer in a subject.

In some embodiments, the inhibitor of SEPHS2 is a CRISPR/Cas9 complex targeting a SEPHS2 gene that is formulated to be delivered via AAV or as a ribonucleoprotein complex, and/or is formulated to be delivered with selenite.

In some embodiments, the cancer is a brain cancer, breast cancer, or renal cancer.

In some embodiments, the subject has a level of SLC7A11 in the cancer above a reference level.

The present methods and compositions can include the use of selenate in addition to or in place of selenite.

Also provided herein are methods for detecting hydrogen selenide gas in a sample. The methods include providing a sample suspected of comprising or producing hydrogen selenide gas; contacting the sample with a composition comprising a detection reagent selected from the group consisting of a metal chloride, nitrate or acetate, wherein the metal is preferably selected from the group consisting of Pb2+, Ag2+, Cd2+, Cu2+, Hg2+, Pb2+ and Zn2+; and detecting the presence of hydrogen selenide gas by measuring a change in the detection reagent.

In some embodiments, the detection reagent is embedded in a matrix, e.g., a matrix that comprises gelatin, starch, polyethylene glycol, or polyvinylpirrolidone (PVP).

In some embodiments, detecting the presence of hydrogen selenide gas comprises using inductively coupled plasma-mass spectrometry (ICP-MS).

In some embodiments, detecting the presence of hydrogen selenide gas comprises measuring a change in the color of the detection reagent.

In some embodiments, the sample is in a multiwell plate, and the detection reagent is present on a cover of the plate.

In some embodiments, the sample is a biological sample, e.g., a sample comprising cultured cells, e.g., cultured cancer cells.

Also provided herein are multiwell plates for use in a method of detecting presence or production of hydrogen selenide gas comprising a plurality of wells and a cover, wherein the cover comprises a coating comprising a detection reagent selected from the group consisting of a metal nitrate, chloride, or acetate, wherein the metal is preferably selected from the group consisting of Pb2+, Ag2+, Cd2+, Cu2+, Hg2+, Pb2+ and Zn2+, wherein the detection reagent is embedded in a matrix.

In some embodiments, the matrix comprises gelatin, starch, polyethylene glycol, or polyvinylpirrolidone (PVP).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D describe the endotoxome platform and identification of SEPHS2 as a putative detoxifying enzyme required in cancer cells. (A) Definition of the endotoxome and putative detoxifying enzymes. (B) Simplified overview of pilot CRISPR/Cas9 screen of detoxifiers. (C) Western Blot showing effective knockout of SEPHS2 gene expression using the pLENTICRISPR V2 lentiviral transduction system. (D) Relative viability following SEPHS2 KO with the different guides across the 12 cell lines. Red dotted line indicates viability at half of that of the control KO condition (black bars, set at 1.0), our cutline for toxicity.

FIGS. 2A-D provide evidence that SEPHS2 plays a detoxifying role in U251 GBM cell line. (A) Knockdown of either SEPHS2 or SEPSECS can block selenoprotein expression. (B) However, only SEPHS2 KO has significant toxicity. (C) Model for ‘pushing’ toxicity in which if SEPHS2 plays a detoxifying role, its KO should synergize with increased selenited input. (D) Synergy is indeed seen with 12 uM selenite treatment in conjunction with SEPHS2 KO. Growth is normalized to the already lower growth rate of the SEPHS2 KO cells, thus the bars in asterisks indicate (statistically significant) synergy.

FIG. 3 shows total selenium levels in standard DMEM/IFS media, human serum, and selenite supplemented media more closely mimics physiological selenium levels.

FIGS. 4A-F show that SLC7A11 mediates selenite import and drives the demand for SEPHS2. (A) Sensitivity to toxic levels of selenite and sensitivity to SEPHS2 KO correlate. (B) KO of SLC7A11 rescues against the toxicity of 12 μM selenite. (C) The group of cell lines that are sensitive to SEPHS2 KO toxicity express higher levels of SLC7A11. (D) SLC7A11 KO prevents intracellular accumulation of selenium following administration of selenite. (E) SLC7A11 KO decreases expression of the selenoprotein GPX4. (F) SLC7A11 KO protects against the toxicity of SEPHS2 KO.

FIG. 5 shows that supplementation of selenite to media increases expression of the selenoproteins SEPHS2 and GPX4.

FIG. 6 is a diagram of all possible entry routes for selenium incorporation into the selenocysteine pathway, labeled in blue. Each route can be genetically dissected for their contribution to selenoprotein production as all enzymes/receptors shown are non-redundant and irreplaceable for their function. Meanwhile, enzymes with asterisks have been determined by us to not be essential, allowing their analyses. CTH essentially has not been yet examined.

FIG. 7 is a bar graph showing SEPHS2 KO Toxicity in cancer and immortalized normal cells. Impact of metabolic gene knockout was measured using pLENTICRISPR based lentiviral transduction of Cas9 and guides (either SEPHS2 guide 1, SEPHS2 guide 2, or nontargeting control guide) in five noncancer, immortalized cell lines (MCF10A, CCDC18CO, THLE2, MCF12A, PNT1a), where the U251 glioma cell lines, shown as positive control, was significantly and negatively impacted. Error bars are +/−SEM. SEPHS2 KO did not impact viability of nontransformed, immortalized cell lines.

FIG. 8 is a bar graph showing that SEPHS2 KO sensitizes cells against the toxic effects of hydrogen selenide gas, demonstrating a detoxifying function of SEPHS2. SEPHS2 KO or control KO cells (MDAMB468 and MDAMB231 cell lines) were subjected to hydrogen selenide gas toxicity. The viability of each set of cells was averaged and relative viability of the SEPHS2 KO cells relative to control KO cells for each cell line are shown.

FIG. 9 is an image of an exemplary multiwell plate for use in a method for detecting hydrogen selenide gas. Silver nitrate and lead acetate were embedded in an immobile matrix of polyvinylpyrrolidone in water and spotted or smeared on the lid of a 96 well plate at specified locations. As shown, the silver nitrate reactive very strongly and selectively with hydrogen selenide to form a brown product, presumably silver selenide. Thus, this method can be used to measure hydrogen selenide gas that is being formed in a well that is placed below or in close proximity to the polyvinylpyrrolidone spot.

### DETAILED DESCRIPTION

Selenium is a trace nutrient that is required by cells in small amounts, but is toxic at high concentrations. It is incorporated into a metabolic pathway that synthesizes the “21st” amino acid selenocysteine, a required component of selenoproteins including proteins with important antioxidant functions such as glutathione peroxidases and thioredoxin reductases.

Dietary forms of selenium such as selenite are known to preferentially accumulate in tumors, and high doses of these compounds can kill cancer cells. However, it is not known why this occurs, and both clinical trials and epidemiological studies involving selenium have not yielded clear results. This may be due to a lack of understanding of how and why selenium is metabolized by normal and cancerous cells.

Selenocysteine Metabolism in Cancer

It was recognized in the 1960's that radioactive selenite (a common form of selenium encountered in the diet) preferentially localizes to tumors¹, and various forms of selenium administered at supraphysiological levels have demonstrated toxicity to cancer cells in cell culture and animal models²⁻⁴. However, this has not led to clinical success, despite dozens of clinical trials involving various forms of selenium administered to patients with various cancers⁵. Furthermore, numerous epidemiological studies have been carried out and have yielded conflicting results pertaining to the relationship between selenium consumption and cancer risk⁶. Thus, while there are intriguing links between selenium and cancer, the exact relationship was unclear and the underlying mechanisms are poorly understood. The metabolic pathway that utilizes selenium—the selenocysteine biosynthesis pathway, including enzymes SEPHS2 and SEPSECS—has not been examined previously in the context of cancer.

As shown herein, disrupting an enzyme in the selenocysteine biosynthesis pathway—selenophosphate synthetase (SEPHS2) is toxic to a subset of cancer cells. SEPHS2 disruption was achieved by CRISPR/Cas9 disruption which is delivered via lentivirus, and this killed cancer cells via toxic selenium accumulation as the selenium cannot be processed into selenoproteins. Importantly, only cells that express a high level of SLC7A11, identified herein as being necessary for import of inorganic selenium into cells, can be targeted this way, and this works as a chemotherapy because several types of cancers typically overexpress SLC7A11 relative to normal cells. Even without SEPHS2 disruption, cancer cells expressing high levels of SLC7A11 were much more susceptible to toxicity of high dose treatment of selenite, a major inorganic form of selenium encountered in the diet. Furthermore, combining SEPHS2 KO with selenite supplementation in cancer cells had a strong synergistic effect compared to either treatment alone.

Thus, several methods are described herein, including methods in which targeted SEPHS2 disruption/inhibition and/or administration of selenite are used, e.g., in cancers that express SLC7A11. In some embodiment, the cancer is a brain cancer, e.g., a glioma; breast cancer; or renal cancer.

Method 1. Targeting SEPHS2 in Cancer Using Inhibitory Nucleic Acids

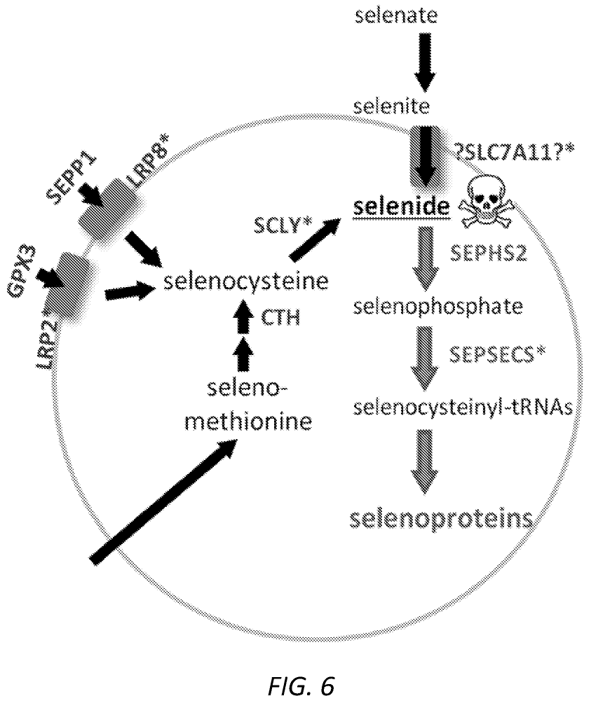

The present methods can include the administration of inhibitors, e.g., inhibitory nucleic acids or CRISPR/Cas9 targeting SEPHS2. As shown in FIG. 6, SEPHS2 catalyzes the production of monoselenophosphate from selenide and ATP. MSP is the selenium donor required for synthesis of selenocysteine. Exemplary human sequences for SEPHS2 (also known as selenide, water dikinase 2) can be found in GenBank at Acc. No. (NM_012248.3, mRNA) and NP_036380.2 (protein). An exemplary human genomic sequence is at Acc No. NC_000016.10 (Range 30443625-30445975, complement) Reference GRCh38.p7 Primary Assembly.

Inhibitory Nucleic Acids Targeting SEPHS2

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target SEPHS2 nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc Natl Acad Sci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261 :1411-1418; Szostak, 1993, TIM 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 mM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Ørom et al., Gene. 2006 May 10; 372( ):137-41). Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜O˜CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623, 070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)nCH₃, O(CH₂)nNH₂ or O(CH₂)nCH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalkylamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. Nos. 3,687,808, as well as 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′ -oxygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

Nucleic Acids Encoding a CRISPR SEPHS2 Gene Editing Complex

The present methods include the delivery of nucleic acids encoding a CRISPR SEPHS2 gene editing complex. The gene editing complex includes a Cas9 editing enzyme and one or more guide RNAs directing the editing enzyme to SEPHS2.

Guide RNAs Directing the Editing Enzyme to SEPHS2

The gene editing complex also includes guide RNAs directing the editing enzyme to SEPHS2, i.e., comprising a sequence that is complementary to the sequence of a nucleic acid encoding SEPHS2, and that include a PAM sequence that is targetable by the co-administered Cas9 editing enzyme. In some embodiments, the precursor sequence is targeted by the guide RNA, i.e., comprising a sequence that is complementary to the sequence of a nucleic acid encoding SEPHS2. In some embodiments, the precursor sequence is targeted by the guide RNA.

Cas9 Editing Enzymes

The methods include the delivery of Cas9 editing enzymes to the cells. The editing enzymes can include one or more of Streptococcus thermophilus (ST) Cas9 (StCas9); Treponema denticola (TD) (TdCas9); Streptococcus pyogenes (SP) (SpCas9); Staphylococcus aureus (SA) Cas9 (SaCas9); or Neisseria haracteriza (NM) Cas9 (NmCas9), as well as variants thereof that are at least 80%, 85%, 90%, 95%, 99% or 100% identical thereto that retain at least one function of the parent protein, e.g., the ability to complex with a gRNA, bind to target DNA specified by the gRNA, and alter the sequence of the target DNA. Variants include the SpCas9 D1135E variant; SpCas9 VRER variant; SpCas9 EQR variant; and the SpCas9 VQR variant, among others.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

The sequences of the Cas9s are known in the art; see, e.g., Kleinstiver et al., Nature. 2015 Jul. 23; 523(7561): 481-485; WO 2016/141224; U.S. Pat. No. 9,512,446; US-2014-0295557; WO 2014/204578; and WO 2014/144761. The methods can also include the use of the other previously described variants of the SpCas9 platform (e.g., truncated sgRNAs (Tsai et al., Nat Biotechnol 33, 187-197 (2015); Fu et al., Nat Biotechnol 32, 279-284 (2014)), nickase mutations (Mali et al., Nat Biotechnol 31, 833-838 (2013); Ran et al., Cell 154, 1380-1389 (2013)), FokI-dCas9 fusions (Guilinger et al., Nat Biotechnol 32, 577-582 (2014); Tsai et al., Nat Biotechnol 32, 569-576 (2014); WO2014144288).

The SpCas9 wild type sequence is as follows:

(SEQ ID NO: 1)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NEKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNWRQLLNAKLITQRKFDNLT
KAERGGLSELDKAGFIKRQLVETRQITKHVAQILDSRNINTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The SaCas9 wild type sequence is as follows:

(SEQ ID NO: 2)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENNINDKRPPRIIKTIASKTQSIKKYSTDILGNLYEVKSKKHPQI
IKKG

See also Hou, Z. et al. Efficient genome engineering in human pluripotent stem cells using Cas9 from Neisseria haracteriza. Proc Natl Acad Sci USA (2013); Fonfara, I. et al. Phylogeny of Cas9 determines functional exchangeability of dual-RNA and Cas9 among orthologous type II CRISPR-Cas systems. Nucleic Acids Res 42, 2577-2590 (2014); Esvelt, K. M. et al. Orthogonal Cas9 proteins for RNA-guided gene regulation and editing. Nat Methods 10, 1116-1121 (2013); Cong, L. et al. Multiplex genome engineering using CRISPR/Cas systems. Science 339, 819-823 (2013); Horvath, P. et al. Diversity, activity, and evolution of CRISPR loci in Streptococcus thermophilus. J Bacteriol 190, 1401-1412 (2008).

As noted above, the Cas9 can be delivered as a purified protein (e.g., a recombinantly produced purified protein, prefolded and optionally complexed with the sgRNA) or as a nucleic acid encoding the Cas9, e.g., an expression construct. Purified Cas9 proteins can be produced using methods known in the art, e.g., expressed in prokaryotic or eukaryotic cells and purified using standard methodology. For example, the methods can include delivering the Cas9 protein and guide RNA together, e.g., as a complex. For example, the Cas9 and gRNA can be can be overexpressed in a host cell and purified, then complexed with the guide RNA (e.g., in a test tube) to form a ribonucleoprotein (RNP), and delivered to cells. In some embodiments, the Cas9 can be expressed in and purified from bacteria through the use of bacterial Cas9 expression plasmids. For example, His-tagged Cas9 proteins can be expressed in bacterial cells and then purified using nickel affinity chromatography. The RNPs can be delivered to the cells in vivo or in vitro, e.g., using lipid-mediated transfection or electroporation. See, e.g., Liang et al., Journal of biotechnology 208 (2015): 44-53; Zuris et al. Nature biotechnology 33.1 (2015): 73-80; Kim et al. Genome research 24.6 (2014): 1012-1019. Efficiency of protein delivery can be enhanced, e.g., using electroporation (see, e.g., Wang et al., Journal of Genetics and Genomics 43(5):319-327 (2016)); cationic or lipophilic carriers (see, e.g., Yu et al., Biotechnol Lett. 2016; 38: 919-929; Zuris et al., Nat Biotechnol. 33(1):73-80 (2015)); or even lentiviral packaging particles (see, e.g., Choi et al., Gene Therapy 23, 627-633 (2016)).

CRISPR Expression Constructs

Expression constructs encoding one or both of guide RNAs and/or Cas9 editing enzymes can be administered in any effective carrier, e.g., any formulation or composition capable of effectively delivering the component gene to cells. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, haracteri S, artificial viral envelopes or other such intracellular carriers, as well as direct injection of the gene construct or CaPO₄ precipitation.

A preferred approach for introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes. These vectors provide efficient delivery of genes into cells, and the transferred nucleic acids are stably integrated into the chromosomal DNA of the host cell. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

As demonstrated herein, a lentiviral CRISPR-Cas9 targeting system provided high and tumor-specific expression of Cas9, the corresponding high SEPHS2 editing efficacy in tumor tissues, while lacking general toxicity or neurotoxicity. Lentiviral vectors transduce dividing as well as quiescent cells. This can be viewed as a major advantage with respect to gene therapy for tumors in general, as within a short treatment window most tumor cells (and especially GSC) do not divide. Therapeutic use of the lentiviral editing approach can be a legitimate alternative to other viral systems, as high viral titers can be produced, nonproliferating cells that are especially abundant in the walls of the tumor cavity after surgery can be transduced, and transduction efficacies are very high. An additional advantage of a locally applied vesicular stomatitis virus glycoprotein (VSV-G) pseudotyped lentivirus is its inactivation by human serum that would reduce systemic effects. To further reduce neurotrophism, and enhance selective tropism for glioma and GSC, the commonly bound envelope glycoprotein of VSV can be replaced with a more selective variant glycoprotein of lymphocytic choriomeningitis virus (LCMV-GP). LCMV-GP is not cytotoxic when injected locally or systemically, can be packaged with other components of the CRISPR-Cas9 system, and efficiently transduces solid glioma tissues as well as infiltrating tumor cells.

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

SEPHS2 genome-editing vectors based on recombinant Adenovirus-5 (Ad5): Ad5 have many advantages for this purpose, including non-integration, lack of insertional mutagenesis, high-efficiency transduction, and accommodation of large expression cassettes; these vectors have also been utilized in multiple clinical trials.

Helper-dependent (HDAd) vectors can also be produced with all adenoviral sequences deleted except the origin of DNA replication at each end of the viral DNA along with packaging signal at 5-prime end of the genome downstream of the left packaging signal. HDAd vectors are constructed and propagated in the presence of a replication-competent helper adenovirus that provides the required early and late proteins necessary for replication.

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. And Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective, easily produced, approved by FDA, and tested in multiple clinical trials, paves new avenues for therapeutic gene editing. Of high relevance to GBM, better tissue distribution of AAV provides an additional advantage for invasive and recurrent tumors. SEPHS2-targeting AAV vectors of various serotypes, including AAV1, AAV2, AAV8, AAV9, and AAVrh.10, can be used, all of which were previously tested in clinical trials. SEPHS2 targeting AAV plasmid [based on Addgene Plasmids #61592, #61594], a single vector expressing SaCas9, gRNA, and Ampicillin selection marker can be utilized. Since PAM consensus sequence is different between SpCas9 and SaCas9 (the late cleaves genomic targets most efficiently with NNGRRT or NNGRR (R=A or G), as also the length required for SaCas9 gRNAs (21-23nt), several targeting constructs have been designed.

Preferably, the CRISPR SEPHS2 editing complex is specific, i.e., induces genomic alterations preferentially at the target site (SEPHS2), and does not induce alterations at other sites, or only rarely induces alterations at other sites.

Pharmaceutical Compositions

The methods described herein can include the administration of pharmaceutical compositions and formulations comprising inhibitory nucleic acid sequences or CRISPR/Cas9/gRNA complexes designed to target SEPHS2.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krutzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Method 2. Using SLC7A11 as a Biomarker

SLC7A11 is an important marker for cancers that can be targeted via SEPHS2 disruption/inhibition, or simply high dose administration of selenite, or both. Thus, the methods can include determining levels of SLC7A11 in subjects, e.g., in samples obtained from the subjects. These subjects can then be selected for treatment with, and optionally treated with, a method described herein, e.g., administration of selenite or a treatment that targets SEPHS2.

Thus, the methods can include obtaining a sample from a subject, and evaluating the presence and/or level of SLC7A11 in the sample, and comparing the presence and/or level with one or more references, e.g., a control reference that represents a normal level of SLC7A11, e.g., a level in a unaffected subject, and/or a reference that represents a level of SLC7A11 associated with likelihood of response to selenite, e.g., a level in a subject having sufficient SLC7A11 expression levels in the cancer cells to render the cells sensitive to selenite or SPEHS2 targeting. Exemplary human sequences for SLC7A11 (also known as cystine/glutamate transporter) can be found in GenBank at Acc. No. (NM_014331.3, mRNA) and NP_055146.1 (protein). An exemplary human genomic sequence is at Acc No. 1.NC_000004.12 (Range 138164094-138242418, complement) Reference GRCh38.p7 Primary Assembly.

SLC7A11 can also be used, e.g., in a clinical trial for the development of a cancer therapy. In these methods, an SLC7A11 level is measured at the initiation of the clinical trial to provide an indication of likely treatment effect or a baseline value to track treatment effect. A biomarker below a reference level at baseline indicates the potential for or likelihood of treatment response, and in some embodiments, the methods can include categorizing or stratifying the subject based on a SLC7A11 level, or selecting the subject for inclusion in or exclusion from the trial. In some embodiments, during or after administration of a treatment under the clinical trial, e.g., at one or more of 1, 2, 3, 4, 5, 6, 8, 9, 10, 11, and/or 12 weeks, or 30, 60, and/or 90 days, or quarterly (e.g., 1, 2, 3, and/or 4 quarters) after initiation of the treatment, the level of SLC7A11 can be measured again, and the value compared to the baseline or an earlier value.

As used herein the term “sample”, when referring to the material to be tested for the level of SLC7A11, means any sample comprising cancer cells from the subject, e.g., cancer cells obtained by or for biopsy, e.g., punch biopsy, needle biopsy, tissue biopsy, or tissue obtained during resection. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of SLC7A11 protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; 6,225,047)

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of SLC7A11. Measurement of the level of a biomarker can be direct or indirect. For example, the abundance levels of SLC7A11 can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such GAPDH and ACTB, are most commonly used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, the presence and/or level of SLC7A11 is above the reference level of the protein(s), then the subject is likely to be sensitive to (i.e., to respond to) a treatment described herein. In some embodiments, the presence and/or level of SLC7A11 is below the reference level, then the subject has a decreased likelihood of responding, and a different treatment should be administered, e.g., a treatment that does not include selenite or targeting SEPHS2. In some embodiments, once it has been determined that a person is likely to respond, then a treatment as described herein, e.g., a treatment that does not include selenite or targeting SEPHS2, can be administered.

Suitable reference values can be determined using methods known in the art, e.g., using standard clinical trial methodology and statistical analysis. The reference values can have any relevant form. In some cases, the reference comprises a predetermined value for a meaningful level of SLC7A11, e.g., a control reference level that represents a level of SLC7A11 in a subject who is expected to respond to a treatment described herein.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the likelihood of response (or response rate) in another defined group. It can be a range, for example, where a population of subjects (e.g., control subjects) is divided equally (or unequally) into groups, such as a low-likelihood group, a medium-likelihood group and a high-likelihood group, or into quartiles, the lowest quartile being subjects with the lowest likelihood and the highest quartile being subjects with the highest highest likelihood, or into n-quantiles (i.e., n regularly spaced intervals) the lowest of the n-quantiles being subjects with the lowest likelihood and the highest of the n-quantiles being subjects with the highest likelihood. Likelihood can be determined based on response rate (with the cohort having the highest response rate being those with the highest likelihood of response).

In some embodiments, the predetermined level is a level or occurrence in the same subject, e.g., at a different time point, e.g., an earlier time point.

Thus, in some cases the level of SLC7A11 in a subject being less than or equal to a reference level of SLC7A11 is indicative of a clinical status (e.g., indicative of a low likelihood of response as described herein). In other cases the level of SLC7A11 in a subject being greater than or equal to the reference level of SLC7A11 is indicative of the presence of a high likelihood of response (wherein “high” and “low” likelihood are relative terms meaning that those in the high likelihood group are statistically more likely to respond that those in the low likelihood group). In some embodiments, the amount by which the level in the subject is the more than the reference level is sufficient to distinguish a high subject from a low subject, and optionally is a statistically significantly less than the level in a control subject. In cases where the level of SLC7A11 in a subject being equal to the reference level of SLC7A11, the “being equal” refers to being approximately equal (e.g., not statistically different).

The predetermined value can depend upon the particular population of subjects (e.g., human subjects) or tissue selected. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Method 3. Administering Selenite Combined with SEPHS2 Disruption

As described herein, the combination of targeted SEPHS2 disruption/inhibition with administration of selenite has a strong synergistic effect in killing cancer cells expressing SLC7A11. Thus, the present methods can include the administration of inhibitory nucleic acids targeting SEPHS2 in combination with selenite treatment.

In some embodiments, the methods include administering a total daily dose of 0.5 mg/m² to 10 mg/m² of selenium, e.g., as sodium selenite. This can be taken, e.g., once daily or in two divided doses, e.g., orally or intravenously, e.g., daily infusion or continuous infusion over several days. Methods of using selenite are described, e.g., inorganic selenium (selenate and selenite, WO 2006032074 A1) and selenium nanoparticles (US 20110262564 A1); see also^{7,8}.

Hydrogen Selenide Gas Detection Methods

We devised a method for specifically detecting hydrogen selenide gas using a detection agent. In general, the methods include utilizing a detection agent such as lead acetate and/or silver nitrate, which were previously used to colorimetrically detect hydrogen sulfide (Ahn et al., Spectrochim Acta A Mol Biomol Spectrosc. 2017 Apr. 15; 177:118-124; Esaki et al., J Biol Chem. 1982 Apr. 25; 257(8):4386-91). For example, a detection reagent such as silver nitrate and lead acetate, which have been previously shown to react with hydrogen sulfide gas to form colorimetric (brown) product silver sulfide and lead sulfide, respectively, can be used. Other metals such as Cd2+, Cu2+, Hg2+, Pb2+ and Zn2+ nitrate, chloride, or acetate could be used as detection reagents as they are expected to react similarly and produce a colored product. Typically, a matrix is used, e.g., the detection agent is embedded in an immobilizing matrix. For the matrix, anything that immobilizes the detection reagent without interfering with the colorimetric detection could be used, such as gelatin, starches, or saccharides/sugars such as xylitol or sucrose or lactose. Other synthetic polymers such as polyethylene glycol could be used. In some embodiments, the matrix comprises polyvinylpirrolidone (PVP). Matrices comprising a combination of polymers can also be used, e.g., PVP/Nafion copolymer. In some embodiments, the methods include mixing the detection agent with polyvinylpyrrolidone (e.g., 100 mM silver nitrate or lead acetate, with in 4% polyvinylpyrrolidone, e.g., in water). This mixture is placed, e.g., by spotting or smearing, onto a substrate such as a slide or a plate, e.g., a multiwall plate, e.g., on the lid of a 96 well plate at selected locations. As a control, sodium selenide is mixed, e.g., with 10% HCl, to form hydrogen selenide gas. This method can be used to measure hydrogen selenide gas, e.g., that is present or being formed in a well that is below or in close proximity to the PVP spot.

To increase sensitivity, the selenium content of the PVP-embedded silver nitrate can be measured, e.g., using inductively coupled plasma-mass spectrometry (ICP-MS), to increase sensitivity and allow precise quantification of selenide captured, as a direct readout of hydrogen selenide gas.

These methods, and plates for use in these methods, can be used, e.g., to identify compounds or conditions that modulate (increase or decrease) production of hydrogen selenide gas

### EXAMPLES

The invention is further described in the following examples, which do not limit the scope of the invention described in the claims.

### Example 1

### Constructing the Endotoxome

GBM cells expressing high levels of mitochondrial serine hydroxymethyltransferase (SHMT2), which converts serine to glycine in the mitochondria, can be selectively poisoned by excess mitochondrial glycine accumulation⁹. The present experiments centered on exploiting toxic metabolites produced in cancer cells to expand upon this initial example.

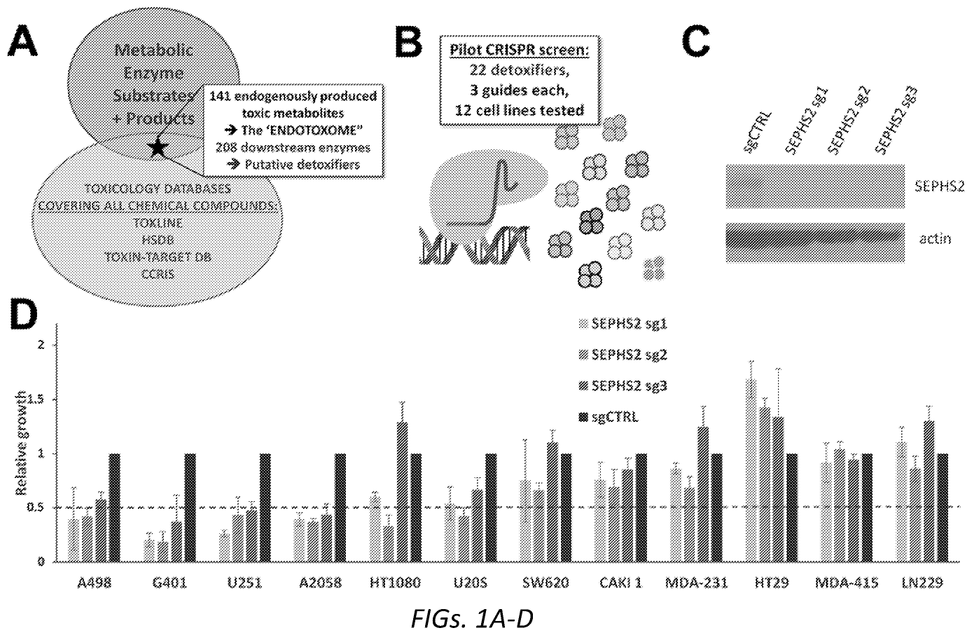

To explore toxic metabolite producing pathways that may be active in glioblastoma, an “endotoxome”, a term we use to denote all metabolites that are endogenously produced in the body that have toxic properties, was manually compiled. The human genome encodes 1,872 metabolic enzymes, and each of their annotated substrates and products was cross referenced with a set of toxicity/chemical safety databases including TOXNET (toxnet.nlm.nih.gov)¹⁰ and Toxin and Target Database (t3db.ca)¹¹, to yield 141 endogenously produced metabolites that have documented toxic properties (FIG. 1A). These toxic metabolites, and the 208 enzymes that metabolize them and thus are putative ‘detoxifiers’, provided a framework for examining toxic metabolite pathways in cancers.

### Example 2

### Pilot Screen Identification of SEPHS2

As an initial pilot the essentiality of 22 of the putative detoxifiers was examined using CRISPR/Cas9 with 3 guides per gene, across a panel of 12 cell lines covering diverse cancers including glioblastoma and triple negative breast cancer. As different cell lines display different infectivity rates and growth rates, and lentiviral CRISPR/Cas9 delivery systems (pLentiCRISPR V2) can be affected by the multiplicity of infection, a method to internally control for different infectivity rates and growth rates of different cell lines was devised and optimized for comparing essentiality across different cell lines. From this pilot screen, selenophosphate synthetase 2 (SEPHS2), a component of the selenocysteine biosynthesis pathway (FIG. 6), emerged as a ‘hit’, as it was essential, but only in half of the 12 cell lines tested (FIG. 1D). This ‘conditional essentiality’ suggested therapeutic potential as it is not an enzyme that is simply essential to all cells, and an opportunity to uncover the mechanisms that determine essentiality based on which cell lines were affected and which cell lines were not.

### Example 3

### SEPHS2 Essentiality is Based on Preventing Selenium Over-Accumulation

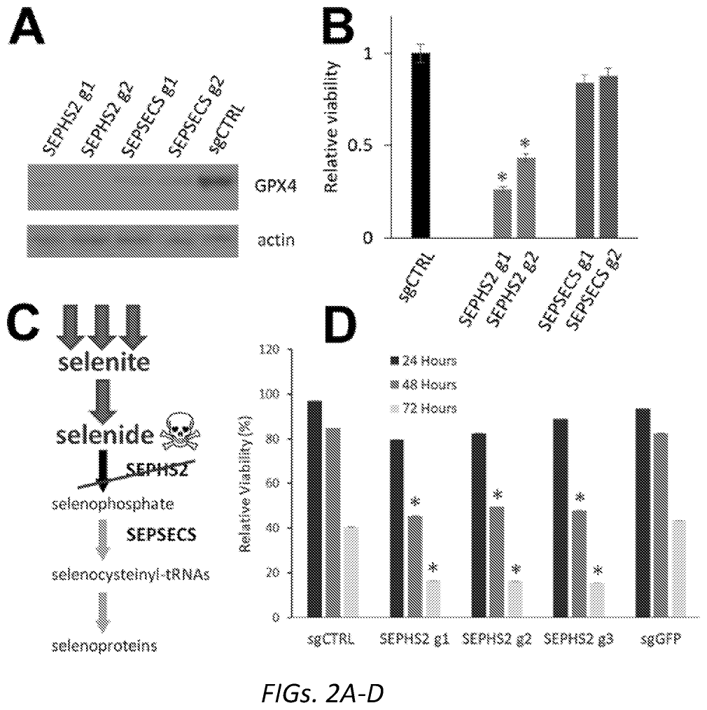

We had classified SEPHS2 as a putative detoxifier as its substrate, selenide, is the most toxic form of selenium¹². The notion of SEPHS2 as a detoxifier has not 1 0 previously been explored, and SEPHS2 is simply known for its enzymatic role (which cannot be replaced by its isoform, SEPHS1¹³) as an intermediate step in producing selenocysteine for selenocysteinyl-tRNA production. The selenocysteine producing pathway is a simple and linear pathway starting from dietary selenite—or selenite or other inorganic forms which can be converted to selenite, selenite being the most common form^{14,15}. Selenite is imported and converted through in an unclear manner to form selenide, which is then metabolized by SEPHS2 to form selenophosphate, accompanied by ATP hydrolysis. The end product of this pathway is selenocysteinyl-tRNA which will be used in the synthesis of 25 selenoproteins, many of which play important antioxidant roles. We set to distinguish whether the acutely toxic effects of SEPHS2 KO on cancer cells was due to selenide detoxification or lack of selenocysteine production. We examined knockdown of a further downstream enzyme, selenocysteine tRNA synthase (SEPSECS). Despite the fact that knockout of SEPHS2 or SEPSECS both impair production of the selenoprotein glutathione peroxidase 1 (GPX4) (FIG. 2A), SEPSECS KO showed only mild effects in impairing cell proliferation in contrast to SEPHS2 KO (FIG. 2B), indicating that lack of selenoprotein production per se is not acutely toxic to cancer cells. On the other hand, we were able to enhance the toxicity of SEPHS2 KO by increasing the selenium availability in the media, via supplementation with selenite, a common form of selenium obtained in the diet (FIGS. 2C and D). This synergistic effect suggested a toxic accumulation mechanism for SEPHS2 KO toxicity, and our collective preliminary findings suggest that SEPHS2 plays a key detoxifying role in cancer cells.

### Example 4

### Cell Culture Conditions do not Accurately Reflect Physiological Selenium Levels

The finding that additional supplementation of selenium to the media enhanced SEPHS2 KO toxicity suggested to us that determining the amount of selenium in the media, and under physiological conditions, is important to predict the efficacy of our strategy.

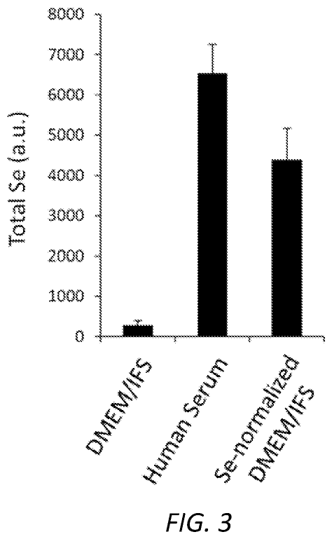

To this end, we measured total selenium levels (as described in^{16,17}) from standard 10% DMEM+10% inactivated fetal bovine serum as well as human serum and found that standard culture conditions underestimate selenium levels by more than 10-fold (FIG. 3). We supplemented selenite to culture media based on previous published selenium measurements, and found that our ‘selenium-normalized’ media displayed total selenium levels much closer to human serum levels. These findings suggest that targeting SEPHS2 in vivo is likely to work even more effectively than in culture due to the higher levels of selenium that are available.

### Example 5

### SLC7A11 Drives Selenium Import and Selenocysteine Metabolism, and Necessitates SEPHS2 Detoxification

We hypothesized that if SEPHS2 is acting as a selenide detoxifier, then its essentiality should depend on the selenide production or import (FIGS. 2C and D). However, it was unclear what upstream transporters and/or enzymes lead to selenide production in cancer cells that would drive the need for detoxification via SEPHS2.

Several putative transporters (e.g., SLC39A8) have been postulated to mediate selenium entry into the cells^{14,18-20}, and additionally there are additional routes such as metabolism of selenomethionine from proteins that can eventually form selenide and thus input into the selenocysteine pathway¹⁴. Selenium is available to cells in a variety of forms (FIG. 6). First, there are three main inorganic forms—selenate (SeO₄²⁻), selenite (SeO₃²⁻), and hydrogen selenide (HSe⁻) (referred to as selenide herein for simplification), which are interchangeable via redox. Of these, selenite is the most abundant and bioavailable form. Cells can also obtain selenium via specific binding and import of two selenocysteine-rich proteins, selenoprotein P (SEPP1) and GPX3, which bind to LRP8 and LRP2, respectively, and can provide selenocysteine upon proteolysis. Importantly, selenocysteine cannot be utilized directly, but must first be converted to selenide via selenocysteine lyase (SCLY) before it can enter the pathway and be incorporated to selenocysteinyl-tRNA. Finally, the majority of selenium in the body is actually present as selenomethionine, where selenomethionine is utilized in place of methionine in roughly 1 out of 1000 residues. However, selenomethionine must be processed to selenocysteine via the transsulfuration pathway, and further metabolized by SCLY, before entering the pathway.

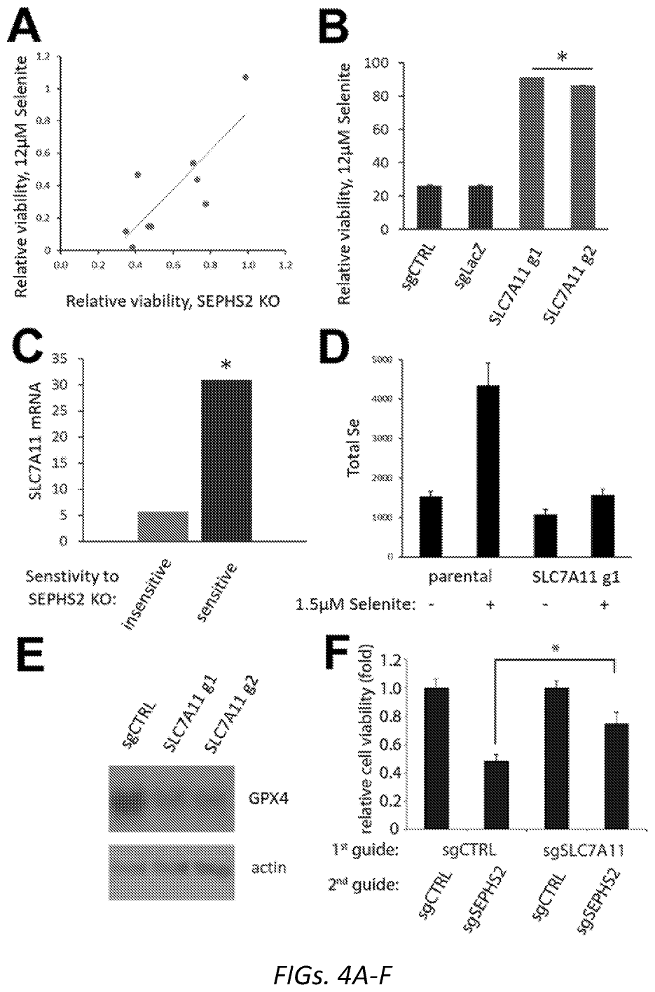

We compared the cells that are sensitive to SEPHS2 KO vs those that are not sensitive to identify key drivers of selenide production. We examined these lines for susceptibility to high, toxic doses of selenite as it is an abundant and bioavailable form of selenium. We observed a strong correlation between susceptibility to high-dose selenite and to SEPHS2 KO (FIG. 4A). This suggests that the SEPHS2 KO-sensitive cell lines are so due to their increased capacity to import selenium in the form of selenite, which would increase detoxification demand. To identify the mediator(s) of selenite import in these cells, we conducted an informed CRISPR/Cas9 screen of 29 candidate selenium transporters: candidates consisted of 1) transporters that were significantly more highly expressed in the SEPHS2 KO group, 2) several transporters previously postulated to mediate selenium import in human cells, and 3) homologs of selenium transporters identified in other species; SLC7A11 fell into groups 1 and 2. Two guides per candidate transporter were knocked out in U251 cells and effects on the toxicity of 12 μM selenite were examined.

As a result of the screen, a single, very robust hit was found—both guides against SLC7A11 resulted in a complete rescue against the toxicity of high-dose selenite (FIG. 4B), while none of the other guides affected selenite toxicity. SLC7A11 was 5.5-fold enriched in SEPHS2-sensitive cell lines (FIG. 4C). Furthermore, knockout of SLC7A11 reduced baseline total intracellular selenium levels, and prevented any increase in total selenium due to supplemented selenite (FIG. 4D). This indicated that SLC7A11 is critical mediator of selenite import in cancer cells. SLC7A11 KO cells also express lower amounts of the selenoprotein GPX4 (FIG. 4E), suggesting that SLC7A11 is responsible for selenocysteine production under standard culture conditions.

To determine whether increased selenium import via SLC7A11 necessitated a requirement for detoxification via SEPHS2, we conducted sequential knockdown experiments using multiple guides. SLC7A11 KO cells, compared to nontargeted control KO cells, were significantly protected against SEPHS2 KO toxicity. These results indicated that SLC7A11 drives selenium import and metabolism that requires detoxification via SEPHS2. The results also defined a causal relationship: If a cell expresses SLC7A11, then SEPHS2 is required.

### Example 6

### Supplementation of Selenite Drives Increased Selenoprotein Expression

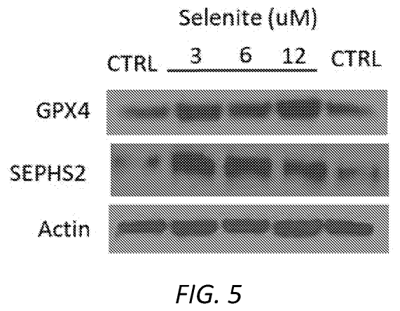

Interestingly, we also noted that supplementation with selenite increased expression of both selenoproteins we examined, GPX4 and SEPHS2 (FIG. 5). This raises the possibility that increased import of selenite via SLC7A11 may play a functional role in cancer cells, by allowing increased expression of selenoproteins such as glutathione peroxidases.

### Example 7

### SLC7A11 is Upregulated in Glioblastoma and Other Cancers

In contrast to SEPHS2, which has not been examined in any cancer, SLC7A11 has actually been widely studied in the context of cancer—as a cystine/glutamate antiporter. SLC7A11 expression and antiporter activity is significantly increased in a number of cancers including glioblastoma, triple negative breast cancer, and lung cancer²¹⁻²⁴. The most established role for SLC7A11 overexpression in cancer cells is that the increased cystine import can fuel increased glutathione production and antioxidant capacity in cancer cells²². Interestingly, SLC7A11 expression can be induced by cytotoxic stresses including radiation and chemotherapeutics, and thus SLC7A11 has been postulated to be a mediator of cancer cell chemoresistance and radioresistance^{25,26}.

These findings indicate that SLC7A11 also mediates selenium import in cancer cells, and thus SLC7A11 overexpressing cancer cells such as in GBM should be selectively dependent on the selenium detoxifying activity of SEPHS2, introducing a therapeutic opportunity.

### Example 8

### SEPHS2 is Selectively Essential to Transformed Cells

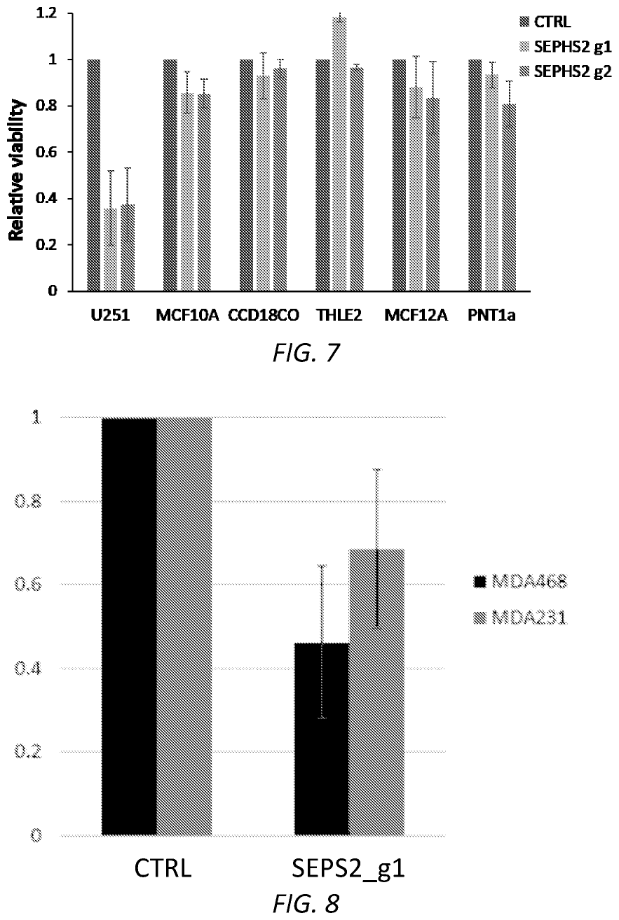

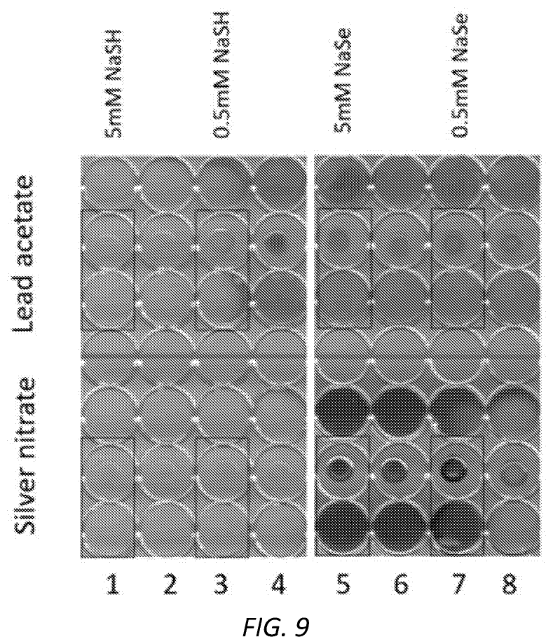

To determining the effects of SEPHS2 KO on viability of nontransformed, immortalized cell lines, as described previously, the impact of metabolic gene knockout was measured using pLENTICRISPR based lentiviral transduction of Cas9 and guides (either SEPHS2 guide 1, SEPHS2 guide 2, or nontargeting control guide). Relative viability, where viability of the control guide group for each cell line was set as 1, was measured using CellTiterGlo system. As shown in FIG. 7, SEPHS2 knockdown did not significantly impair viability in five noncancer, immortalized cell lines (MCF10A, CCDC18CO, THLE2, MCF12A, PNT1a), where the U251 glioma cell lines, shown as positive control in FIG. 7, was significantly and negatively impacted. The results showed that that SEPHS2 KO did not impair the viability of five nontransformed (normal, noncancerous) immortalized cell lines, demonstrating that SEPHS2 is selectively essential to transformed cells (FIG. 7).

### Example 9

### SEPHS2 is Selectively Essential to Transformed Cells

To determine the effects of SEPHS2 KO on tumor formation and tumor growth, orthotopic (mammary fat pad) xenograft assays were used. MDAMB231 cells were subjected to knockout of SEPHS2 via pLENTICRISPR (SEPSH2 guide 1), or to control nontargeting knockout (nontargeting guide). Five days after infection, equal numbers of viable cells from each group were injected and the mice were monitored biweekly. Table 1 shows results for the number of tumors formed (out of 7 injections) and the average size of the formed tumors, at 4 weeks.

| | Avg Tumor | Measurable |
|---|---|---|
| | Volume | Tumor |
| | (mm\hat{ }3) | Formation |
| Tumors formed from Control KO cells | 116.01 | 4 out of 7 |
| Tumors formed from SEPHS2 KO cells | 6.68 | 1 out of 7 |

These results show decreased tumor forming capability and tumor growth of SEPHS2 KO MDA-MB-231 cells when compared to their non-KO controls.

### Example 10

### Hydrogen Selenide Gas Toxicity Assay

We developed a novel gas toxicity assay in which hydrogen selenide was produced in one well of a 96-well plate and the toxic effects were measured in an adjacent well. As described, SEPHS2 KO or control KO was achieved via pLENTICRISPR lentiviral transduction, and then cells were subjected to hydrogen selenide gas toxicity. Hydrogen selenide gas was formed by dissolving sodium selenide in an acidic (10% Hydrochloric Acid) solution in the central wells of a 96-well culture plate. SEPHS2 KO and CTRL cells (for MDAMB468 and MDAMB231 cell lines) were pre-seeded at various distances from the source of hydrogen gas toxicity (distances were equal under both conditions). The viability of each set of cells was averaged and relative viability of the SEPHS2 KO cells relative to control KO cells for each cell line are shown in FIG. 8. Using this assay, we found that SEPHS2 KO cells were hypersensitive to selenide gas, indicating a detoxification function of SEPHS2.

### Example 11

### Hydrogen Selenide Gas Detection Assay

We devised a method for specifically detecting hydrogen selenide gas. This involves utilizing polyvinylpirrolidone (PVP) as a matrix to embed lead acetate and/or silver nitrate, which was previously used to colorimetrically detect hydrogen sulfide (Ahn et al., Spectrochim Acta A Mol Biomol Spectrosc. 2017 Apr. 15; 177:118-124; Esaki et al., J Biol Chem. 1982 Apr. 25; 257(8):4386-91). Briefly, silver nitrate and lead acetate, which have been previously shown to react with hydrogen sulfide gas to form colorimetric (brown) product silver sulfide and lead sulfide, respectively, were embedded in an immobile matrix. 1M silver nitrate, 1M lead acetate, and 5% (w/v) solution of polyvinylpyrrolidone (PVP) were prepared freshly for each experiment. The 1M silver nitrate or 1M lead acetate was mixed with 5% PVP at a ratio of 1:9, yielding final concentrations of 100 mM of silver nitrate or lead acetate, and 4.5% PVP. About 15 ul of this mixture was spotted on the inside of a 96-well plate cover. Alternatively, the 15 ul could be spread across the entire circular surface. The plate was placed above wells in which hydrogen selenide gas was produced, and upon immediate reaction with the selenide gas formed silver selenide or lead selenide, which are brown in color compared to the clear original spot. As a positive control, hydrogen selenide gas was formed by mixing sodium selenide at a final 50 mM concentration, into 3.7% hydrochloric acid solution.

The spots were developed for up to 2 minutes, noting color development, before removing from the hydrogen selenide gas producing plate and immediately imaging.

An exemplary plate is shown in FIG. 8. The location of the well forming the gas is marked with black rectangles, and concentrations of reagents used to create gas are shown. As shown, the silver nitrate reacted very strongly and selectively with hydrogen selenide to form a brown product, presumable silver selenide. Thus, this method can be used to measure hydrogen selenide gas that is being formed in a well placed below or in close proximity to the polyvinylpyrrolidone spot.

We found that this method appears to be more sensitive towards hydrogen selenide than sulfide (FIG. 8).

This data, in addition to the first demonstration of use of these compounds to detect hydrogen sulfide, provides a direct comparison of the performance of silver nitrate versus lead acetate, and shows that silver nitrate displays increased performance in terms of colorimetric reaction. As shown in FIG. 8, comparing 0.5 mM NaSh with 0.5 mM NaSe shows that both probes, but in particular the silver nitrate probe, react selectively with hydrogen selenide as compared to hydrogen sulfide. Thus, surprisingly, under these conditions we saw better performance of silver nitrate in relation to lead acetate, and increased specificity towards hydrogen selenide as opposed to the previously shown use in detecting hydrogen sulfide. It is possible, however, that the rates of hydrogen selenide formation and hydrogen sulfide formation could be different enough to could account for higher signal for the probes against hydrogen selenide.

### REFERENCES

1 Cavalieri, R. R., Scott, K. G. & Sairenji, E. Selenite (75Se) as a tumor-localizing agent in man. J Nucl Med 7, 197-208 (1966).
2 Husbeck, B., Nonn, L., Peehl, D. M. & Knox, S. J. Tumor-selective killing by selenite in patient-matched pairs of normal and malignant prostate cells. Prostate 66, 218-225, doi:10.1002/pros.20337 (2006).
3 Lunoe, K. et al. Investigation of the selenium metabolism in cancer cell lines. Metallomics 3, 162-168, doi:10.1039/c0mt00091d (2011).
4 Zeng, H., Cheng, W. H. & Johnson, L. K. Methylselenol, a selenium metabolite, modulates p53 pathway and inhibits the growth of colon cancer xenografts in Balb/c mice. J Nutr Biochem 24, 776-780, doi:10.1016/j.jnutbio.2012.04.008
S0955-2863(12)00121-0 [pii] (2013).
5 Sanmartin, C., Plano, D., Font, M. & Palop, J. A. Selenium and clinical trials: new therapeutic evidence for multiple diseases. Curr Med Chem 18, 4635-4650, doi:BSP/CMC/E-Pub/2011/339 [pii] (2011).
6 Clark, L. C. The epidemiology of selenium and cancer. Fed Proc 44, 2584-2589 (1985).
7 Yang, Y. et al. The anticancer effects of sodium selenite and selenomethionine on human colorectal carcinoma cell lines in nude mice. Oncol Res 18, 1-8 (2009).
8 Hui, K. et al. The p38 MAPK-regulated PKD1/CREB/Bcl-2 pathway contributes to selenite-induced colorectal cancer cell apoptosis in vitro and in vivo. Cancer Lett 354, 189-199, doi:10.1016/j.canlet.2014.08.009
S0304-3835(14)00437-6 [pii] (2014).
9 Kim, D. et al. SHMT2 drives glioma cell survival in ischaemia but imposes a dependence on glycine clearance. Nature 520, 363-367, doi:10.1038/nature14363
nature14363 [pii] (2015).
10 Wexler, P. The framework of toxicology information. Toxicology 60, 67-98, doi:0300-483X(90)90164-C [pii] (1990).
11 Wishart, D. et al. T3DB: the toxic exposome database. Nucleic Acids Res 43, D928-934, doi:10.1093/nar/gku1004
gku1004 [pii] (2015).
12 Lockitch, G. Selenium: clinical significance and analytical concepts. Crit Rev Clin Lab Sci 27, 483-541, doi:10.3109/10408368909114596 (1989).
13 Xu, X. M. et al. Selenophosphate synthetase 2 is essential for selenoprotein biosynthesis. Biochem J 404, 115-120, doi:BJ20070165 [pii]
10.1042/BJ20070165 (2007).
14 Burk, R. F. & Hill, K. E. Regulation of Selenium Metabolism and Transport. Annu Rev Nutr 35, 109-134, doi:10.1146/annurev-nutr-071714-034250 (2015).
15 Hatfield, D. L., Tsuji, P. A., Carlson, B. A. & Gladyshev, V. N. Selenium and selenocysteine: roles in cancer, health, and development. Trends Biochem Sci 39, 112-120, doi:10.1016/j.tibs.2013.12.007
S0968-0004(13)00209-0 [pii] (2014).
16 Watkinson, J. H. Fluorometric determination of selenium in biological material with 2,3-diaminonaphthalene. Anal Chem 38, 92-97 (1966).
17 Sheehan, T. M. & Gao, M. Simplified fluorometric assay of total selenium in plasma and urine. Clin Chem 36, 2124-2126 (1990).
18 McDermott, J. R. et al. Zinc- and bicarbonate-dependent ZIP8 transporter mediates selenite uptake. Oncotarget 7, 35327-35340, doi:10.18632/oncotarget.9205
9205 [pii] (2016).
19 Rosen, B. P. & Liu, Z. Transport pathways for arsenic and selenium: a minireview. Environ Int 35, 512-515, doi:10.1016/j.envint.2008.07.023
S0160-4120(08)00148-7 [pii] (2009).
20 Tobe, T. et al. Selenium uptake through cystine transporter mediated by glutathione conjugation. J Toxicol Sci 42, 85-91, doi:10.2131/jts.42.85 (2017).
21 Briggs, K. J. et al. Paracrine Induction of HIF by Glutamate in Breast Cancer: EglN1 Senses Cysteine. Cell 166, 126-139, doi:10.1016/j.cell.2016.05.042
S0092-8674(16)30593-1 [pii] (2016).
22 Conrad, M. & Sato, H. The oxidative stress-inducible cystine/glutamate antiporter, system x (c) (-): cystine supplier and beyond. Amino Acids 42, 231-246, doi:10.1007/s00726-011-0867-5 (2012).
23 Takeuchi, S. et al. Increased xCT expression correlates with tumor invasion and outcome in patients with glioblastomas. Neurosurgery 72, 33-41; discussion 41, doi:10.1227/NEU.0b013e318276b2de (2013).
24 Timmerman, L. A. et al. Glutamine sensitivity analysis identifies the xCT antiporter as a common triple-negative breast tumor therapeutic target. Cancer Cell 24, 450-465, doi:10.1016/j.ccr.2013.08.020
S1535-6108(13)00366-8 [pii] (2013).
25 Huang, Y., Dai, Z., Barbacioru, C. & Sadee, W. Cystine-glutamate transporter SLC7A11 in cancer chemosensitivity and chemoresistance. Cancer Res 65, 7446-7454, doi:65/16/7446 [pii]
10.1158/0008-5472.CAN-04-4267 (2005).
26 Zhang, P. et al. xCT expression modulates cisplatin resistance in Tca8113 tongue carcinoma cells. Oncol Lett 12, 307-314, doi:10.3892/01.2016.4571
OL-0-0-4571 [pii] (2016).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a cancer in a subject, the method comprising administering to the subject an inhibitor of SEPHS2, wherein the inhibitor of SEPHS2 is an inhibitory nucleic acid, preferably selected from the group consisting of an antisense, siRNA, or LNA targeting a SEPHS2 nucleic acid, and a CRISPR/Cas9 complex targeting a SEPHS2 gene.

2. The method of claim 1, wherein the inhibitory nucleic acid is a CRISPR/Cas9 complex targeting a SEPHS2 gene delivered via AAV or as a ribonucleoprotein complex.

3. The method of claim 1, wherein the cancer is a brain cancer, breast cancer, or renal cancer.

4. The method of claim 1, further comprising administering a treatment comprising administration of selenite to the subject.

5. A method of determining whether a subject who has cancer is likely to respond to a treatment comprising administration of selenite, and optionally selecting a subject who has cancer for treatment with selenite, the method comprising:
determining a level of SLC7A11 expression in a sample comprising cancer cells from the subject;
comparing the level of SLC7A11 in the sample to a reference level, wherein the presence of a level of SLC7A11 in the sample above the reference level indicates that the subject is likely to respond to a treatment comprising administration of selenite;
and optionally selecting the subject for a treatment comprising administration of selenite.

6. The method of claim 5, further comprising administering a treatment comprising administration of selenite to the subject who has a level of SLC7A11 in the sample above the reference level.

7. The method of claim 6, further comprising administering to the subject an inhibitor of SEPHS2, wherein the inhibitor of SEPHS2 is an inhibitory nucleic acid, preferably selected from the group consisting of an antisense, siRNA, or LNA targeting a SEPHS2 nucleic acid, and a CRISPR/Cas9 complex targeting a SEPHS2 gene.

8. The method of claim 5, wherein the cancer is a brain cancer, breast cancer, or renal cancer.

9. (canceled)

10. (canceled)

11. (canceled)

12. (canceled)

13. (canceled)

14. A method of detecting hydrogen selenide gas in a sample, the method comprising:
providing a sample suspected of comprising or producing hydrogen selenide gas;
contacting the sample with a composition comprising a detection reagent selected from the group consisting of a metal nitrate, chloride, or acetate, wherein the metal is preferably selected from the group consisting of Pb2+, Ag2+, Cd2+, Cu2+, Hg2+, Pb2+ and Zn2+; and
detecting the presence of hydrogen selenide gas by measuring a change in the detection reagent.

15. The method of claim 14, wherein the detection reagent is embedded in a matrix.

16. The method of claim 14, wherein the matrix comprises gelatin, starch, polyethylene glycol, or polyvinylpirrolidone (PVP).

17. The method of claim 14, wherein detecting the presence of hydrogen selenide gas comprises using inductively coupled plasma-mass spectrometry (ICP-MS).

18. The method of claim 14, wherein detecting the presence of hydrogen selenide gas comprises measuring a change in the color of the detection reagent.

19. The method of claim 14, wherein the sample is in a multiwell plate, and the detection reagent is present on a cover of the plate.

20. The method of claim 14, wherein the sample is a biological sample, preferably a sample comprising cultured cells, optionally cultured cancer cells.

21. A multiwell plate for use in a method of detecting presence or production of hydrogen selenide gas comprising a plurality of wells and a cover, wherein the cover comprises a coating comprising a detection reagent selected from the group consisting of a metal nitrate, chloride, or acetate, wherein the metal is preferably selected from the group consisting of Pb2+, Ag2+, Cd2+, Cu2+, Hg2+, Pb2+ and Zn2+, wherein the detection reagent is embedded in a matrix.

22. The multiwell plate for the use of claim 20, wherein the matrix comprises gelatin, starch, polyethylene glycol, or polyvinylpirrolidone (PVP).


SEQUENCE LISTING (2 sequences)

SEQ ID NO: 1 - protein, 1368 aa, Streptococcus pyogenes [sequence omitted]

SEQ ID NO: 2 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG